SEQUENCE LISTING (6 sequences)

SEQ ID NO: 1 - protein, 115 aa, Artificial Sequence
  synthesized
DIVLTQTPLSLTVSLGDQASISCRSSQSIVHSNGNTYLGWYLQRPGQSPKLLIYKVSNRFSGVPDRFSGSGSGTDFTLKISRVEAEDLGVYYCFQGSHVPLTFGAGTKLELKRAD

SEQ ID NO: 2 - protein, 117 aa, Artificial Sequence
  synthesized
QVQLQESGGGLVKPGGSLKLSCAASGFSFSDYYMYWVRQAPEKRLEWVASISFDGTYTYYTDRVKGRFTISRDNAKNNLYLQMSSLKSEDTAMYYCARDRPAWFPYWGQGTLVTVSA

SEQ ID NO: 3 - protein, 116 aa, Artificial Sequence
  synthesized
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSA

SEQ ID NO: 4 - protein, 107 aa, Artificial Sequence
  synthesized
DIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIK

SEQ ID NO: 5 - protein, 120 aa, Artificial Sequence
  synthesized
QVQLVQSGAEVKKPGASVKVSCKVSGYTFTHYWMHWVRQRPGKGLEWMGEIDPFDSYTYYNQKFKGRVTMTVDTSSDTAYMELSSLRSEDTAVYYCARPLPGTGWYFDVWGQGTTVTVSS

SEQ ID NO: 6 - protein, 108 aa, Artificial Sequence
  synthesized
EIVLTQSPTTLSLSPGERATLSCSASSSISSTYLHWYQQKPGFPPRLLIYGTSNLASGIPACFSGSGSGTDFTLTISSLEAEDFAVYYCQQGSSLPFTFGQGTKLEIK

(19) United States
(12) Patent Application Publication
(10) Pub. No.: US 2021/0116456 A1
(43) Pub. Date: Apr. 22, 2021

(54) COMPOSITIONS AND METHODS FOR DIAGNOSIS AND TREATMENT OF CANCER

(71) Applicant: ARBELE LIMITED, Hong Kong (CN)

(72) Inventors: Donald E. STAUTON, Kirkland, WA (US); John Moonching LUK, BELLEVUE, WA (US)

(21) Appl. No.: 17/055,560

(22) PCT Filed: May 16, 2019
(86) PCT No.: PCT/US2019/032752

§ 371 Date: Nov. 13, 2020

Related U.S. Application Data
(60) Provisional application No. 62/672,319, filed on May 16, 2018.

(51) Int. Cl. G01N 33/574; C07K 16/28

(52) U.S. Cl. CPC G01N 33/57492; C07K 2317/24; G01N 2333/705; C07K 16/28

(57) ABSTRACT

A method for diagnosing CDH17 positive tumor cells and cancer in a subject is disclosed, including but not limited to, the steps of obtaining a sample from the subject; contacting the sample with a capturing antibody to provide a captured sample; contacting the captured sample with a detecting antibody or lipid nanoprobe (LNP) to provide a detecting sample; determining the amount of the detecting antibody or LNP in the detecting sample; and based on the amount of the detecting antibody or LNP, determining the probability of a subject possessing a tumor.

### CROSS REFERENCE TO RELATED APPLICATIONS

This application claims the benefit of the filing date of U.S. Provisional Application Ser. No. 62/672,319 filed May 16, 2018 under 35 U.S.C. 119(e), the entire disclosures of which are incorporated by reference herein.

### TECHNICAL FIELD

The present disclosure relates in general to the field of cancer diagnosis, and more specifically to reagent and method of diagnosing CDH17-positive cancer.

### BACKGROUND

Gastrointestinal (GI) cancers are leading causes of morbidity and mortality worldwide. Colorectal carcinoma (CRC) alone represents approximately 10% of all cancer diagnosis and is the second leading cause of cancer deaths world-wide (Verdaguer 2017). Early detection of localized tumors and ideally in stage 1, can enable curative surgery for most tumors (Siegel 2017). Conventional blood-based tumor marker assays such as CEA and CA19-9 lack the sensitivity and specificity required for early detection of GI cancers (Lech 2016). Although non-invasive blood tests and liquid biopsies (to analyze circulating tumor DNA or ctDNA) have progressed recently, there remains a need to accurately detect and stage a greater percentage of GI cancers, especially those at early stages. For example, a very recent blood test for plasma protein and ctDNA markers, CancerSEEK, has increased the percentage of cancers detected (Cohen 2018). However only around 40% of stage I cancers are detected (20% for esophageal). In general, detecting cancer at early stages by liquid biopsy remains difficult as these tumors do not appear to release a sufficient amount of ctDNA into plasma despite the use of extremely sensitive techniques (Bettegowda 2014, Cohen 2017). Other approved tests, such as biopsy or colonoscopy, are invasive and tissue for biopsy is not always accessible over the course of clinical care. Thus, there is an unequivocal need for a better and more sensitive blood-based biomarker assays to enable early detection of GI cancers.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The present disclosure provides methods for diagnosing a tumor in a subject. In one embodiment, the method includes the steps of obtaining a sample from the subject; contacting the sample with a capturing antibody to provide a captured sample; contacting the captured sample with a detecting antibody or lipid nanoprobe (LNP) to provide a detecting sample; determining the amount of the detecting antibody or LNP in the detecting sample; and based on the amount of the detecting antibody or LNP, determining the probability of a subject possessing a tumor. The capturing antibody may include an anti-CDH17 monoclonal antibody. The anti-CD17 monoclonal antibody may have highly specific binding activity to an exosome, microvesicle, or soluble CDH17 fragment. In one embodiment, the capturing antibody may be a monoclonal antibody having a binding activity to CD9, CD63, CD81, CD45 or a combination thereof. In one embodiment, the detecting antibody may include an antibody having affinity to CDH17, TROP2, CD63, CD9, CD81, CD45, a tumor marker, a tissue marker antibody, or a combination thereof.

In one embodiment, the steps in the method may be in any order. In one embodiment, the steps in the method may be sequential. In one embodiment, two or more steps in the methods may be carried simultaneously. In one embodiment, two or more steps in the methods may happen in one reaction container.

In at least one embodiment, the method may include the steps of obtaining a sample from the subject; contacting the sample with a capturing antibody to provide a captured sample; contacting the captured sample with a detecting antibody or a novel lipid based nanoprobe (LNP) to provide a detecting sample; determining the amount of the detecting antibody or LNP in the detecting sample; and based on the amount of the detecting antibody or LNP, determining the probability of a subject possessing a tumor.

In at least one embodiment, the method includes the steps of obtaining a sample from the subject; contacting the sample with a capturing antibody to provide a captured sample; determining the amount of captured sample; and based on the amount of captured sample, determining the probability of a subject possessing a tumor.

In at least one embodiment, the method includes the steps of obtaining a sample from the subject; labeling the sample with a florescent DNA/RNA stain to provide a labeled sample; contacting the labeled sample with a capturing antibody to provide a captured sample; determine the amount of captured sample; and based on the amount of captured sample, determining the probability of a subject possessing a tumor.

In at least one embodiment, the capturing antibody may include a capturing anti-CDH17 monoclonal antibody. In at least one embodiment, the capturing antibody may include a monoclonal antibody having a binding activity to an exosome, microvesicle or soluble CDH17 fragment. In one embodiment, the capturing antibody may have a binding affinity to CDH17 or a fragment thereof.

In at least one embodiment, the capturing antibody may include a monoclonal antibody having a binding activity to CD9, CD63, CD81, CD45 or a combination thereof.

In at least one embodiment, the detecting antibody may include an antibody having a binding affinity to CDH17, TROP2, CD63, CD9, CD81, CD45, a tumor marker, a tissue marker, or a combination thereof.

In at least one embodiment, the detecting step is carried out by using a novel lipid based nanoprobe (LNP).

In at least one embodiment, the tumor is a CD17 positive tumor. In one embodiment, the tumor includes a cancer of the gastrointestinal system. In at least one embodiment, the tumor includes a colon cancer.

In at least one embodiment, the sample includes a bodily fluid. In one embodiment, the bodily fluid comprises blood.

The disclosure further provides methods for assay development. In one embodiment, three platforms were developed and used for a comparison of the most robust assay, including proximity luminescence, ELISA, and flow cytofluorometric analysis. CDH17 capture and detection antibodies are used to screen from a large panel of anti-CDH17 antibodies for one or more optimized combinations for the highest level of sensitivity. To increase the sensitivity of any diagnostic assay further, functionally orientated recombinant CDH17-capturing antibodies were generated. In one embodiment, the efficiency of a novel lipid based nanoprobe (LNP) was developed and compared with above-mentioned assays for capturing and detection of CDH17 EV. In one embodiment, assays were developed for detecting and quantification of the levels of cCDH17, CDH17 EV, and total blood CDH17, respectively.

In one embodiment, the application provides methods for screening and diagnosing biological samples from patients. A large panel of patients and normal blood samples (plasma/sera) were diagnosed and compared using the novel and optimized assays described herein. In one embodiment, blood samples from patients with gastroenteritis, pancreatitis, and inflammatory bowel disease (IBD) were tested to determine if CDH17 in blood increases in non-cancer inflammatory diseases involving GI tissue. In one embodiment, the cancer being diagnosed is colorectal cancer (CRC). In one embodiment, the endpoint for clinical sample validation was the demonstration of a statistically significant increase in sCDH17, CDH17 EV or total CDH17 in GI patient blood. In another embodiment, endpoints include the demonstration of a significant increase in CDH17 blood levels with increasing tumor stages and/or any decrease with post-treatment.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 depicts the characterization of CDH17 expression in the samples from CRC patients at stage I-IV by counting CDH17 positive immunohistochemical staining (A) and CDH17 specific plasma marker units (B);

FIG. 2 depicts the measurement of CDH17 protein concentration in the serum samples from CRC patients at stage I-III;

FIG. 3 illustrates that the level of CDH17 positive circulating tumor cells (CTC) in individual CRC patients increases with tumor stage and decreases 5 days post-surgery with sample slides from a CRC patient blood specimen;

FIG. 4 depicts the expression of CDH17 on exosomes purified by ultracentrifugation from tumor cell line culture media;

FIG. 5 illustrates the concentration of CDH17 in cancer cell culture media (A) and in CRC plasma (B) by ELISA;

FIG. 6 illustrates three assay platforms, fluorescent ELISA, flow cytofluorimetry, and proximity luminescence, to quantitate CDH17 EVs in liquid samples (A, B, and C); captured CDH17 EVs (D, E, and F); and other proteins on CDH17 EVs (G, H, and I);

FIG. 7 reveals examples of CDH17 monoclonal antibodies specific for different CDH17 ectodomains; and

FIG. 8 depicts the standardization and sensitivity of assays for quantifying captured CDH17 by flow cytometry (upper) and/or ELISA (lower). The standard curve may be established by using recombinant CDH17 in the form of either captured on beads or wells coated with one or more CDH17 monoclonal antibodies. The detection agents include detecting antibodies, such as a different CH17 monoclonal antibody. The sensitivity of assays is about 400 to 500 pg/mL.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions and methods related to cancer diagnosis.

CDH17 is an oncogene and cell adhesion membrane protein with restricted expression in normal GI tissue (Liu 2009, Wang 2013). CDH17 is expressed at high levels and in a high percentage of tumors in patients with colorectal carcinoma (>95%), gastric adenocarcinoma (90%) and esophageal adenocarcinoma (82%) (Altree-Tacha 2017; Ordonez 2014; Matsusaka 2016; Panarelli 2012; Su 2008). The level of CDH17 expression as measured by a cDNA microarray seems to be increased in precancerous tissue, such as pre-gastric cancer intestinal metaplasia (IM) and spasmolytic polypeptide-expressing metaplasia (SPEM) (Lee H J et al 2010). However, there is currently no quantification of levels of CDH17 expression in their association with the types and/or stages of GI tumors. As a result of extensive research, this disclosure provides, among others, compositions, reagents and methods for quantifying CDH17 expressions in tumors with surprising accuracy and sensitivity.

CDH17 is expressed at high levels in different types of GI cancer. Using the cancer genome atlas (TCGA) RNA sequencing data (RNA Seq V2), the level of CDH17 expression in different types of malignancies can be ranked from low to high. The high levels of CDH17 expression is associated with GI cancers, including without limitation, colorectal, gastric, pancreatic, and esophageal cancer. In addition, the level of CDH17 expression is found to be high in papillary renal cell carcinoma (PRCC) and cholaniocarcinoma.

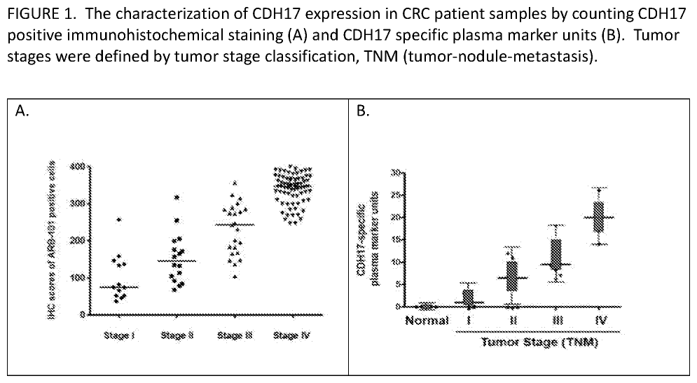

The expression of CDH17 in the majority of GI cancer can be determined by immunohistochemistry (IHC). Approximately 100% of colorectal, 90% of gastric adenocarcinoma and 82% of esophageal adenocarcinoma express CDH17. A correlation between the level of CDH17 expression in CRC and the cancer stage I to IV is shown in FIG. 1-3.

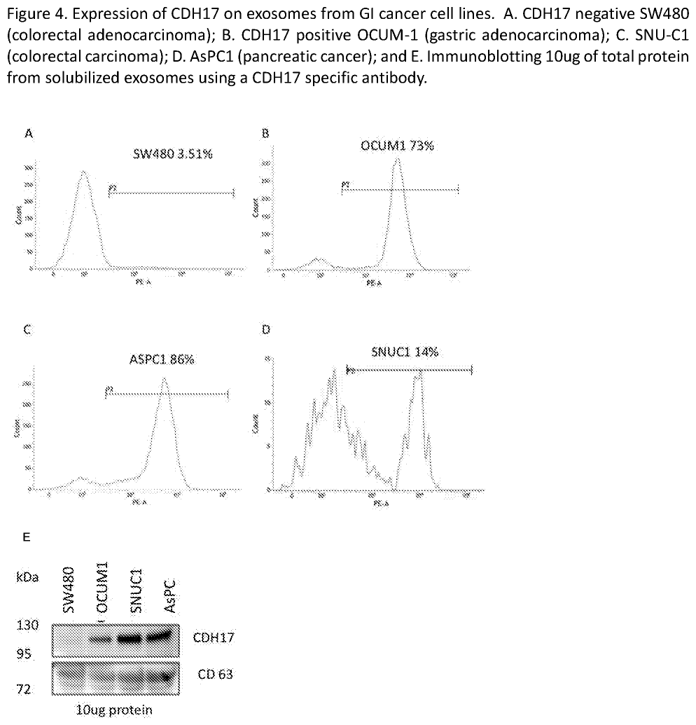

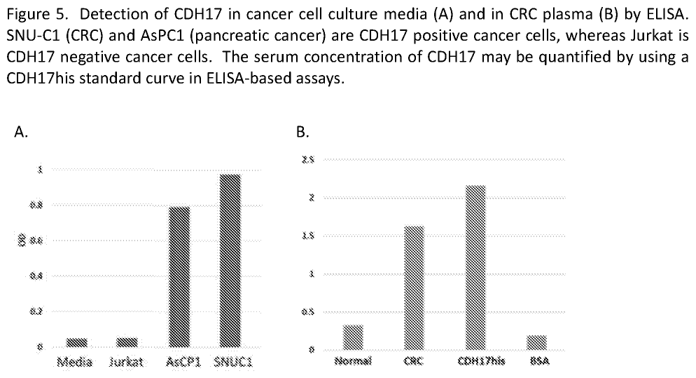

The present disclosure relates to development of a sensitive and specific assay for CDH17 in blood. In one embodiment, the assay (s) disclosed herein are useful for clinical sample validation. Both cancer cell culture media and patient blood samples were used for development, validation, and optimization of assays. Both sCDH17 and CDH17EV were readily detectable from cultured cancer cell media (FIG. 4-5). However, the cleaved forms of sCDH17 and potentially CDH17 on EV membranes in cancer cell culture media or patient blood could be different, namely, partial CDH17 comprising one or more but not all epitopes. Thus, the emphasis is placed on identifying an array of antibodies that can capture all forms of CDH17 from patient samples.

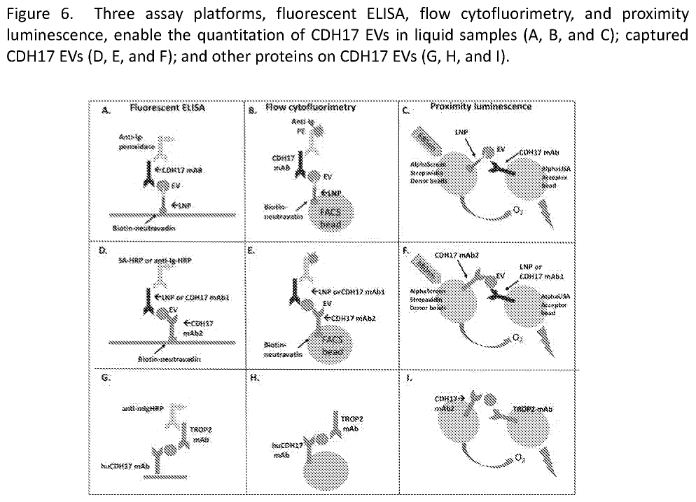

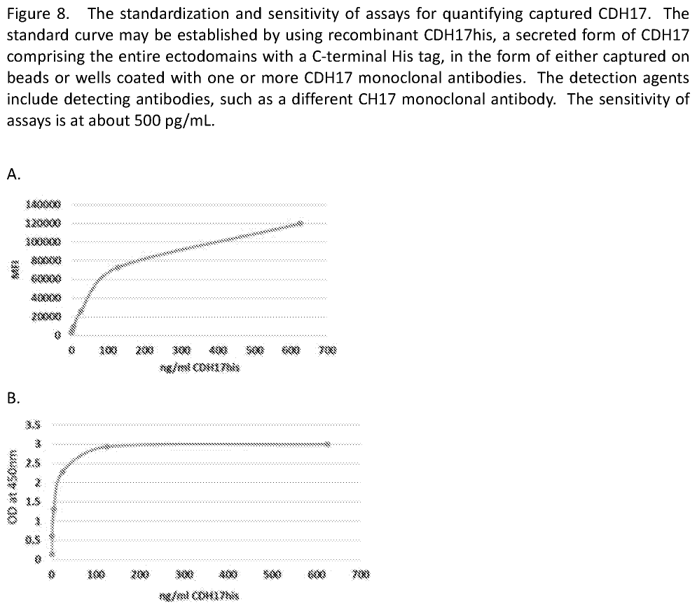

Three platforms, proximity based chemiluminescence, ELISA and flow cytofluorometry as shown in FIG. 6, were compared to develop assays with the greatest sensitivity and dynamic range for sCDH17, CDH17EV, and total CDH17. Proximity luminescence has an advantage with a shorter assay time due to fewer steps. It also can enable very sensitive assays requiring very small analyte volumes (Yoshioka 2014). The assay captured sCDH17 and/or CDH17EV via immobilized CDH17 antibodies or LNP (specific for EV). Captured CDH17 were measured using functionally orientated non-competitive CDH17 antibodies (FIG. 8) or LNP (specifically detecting EV). An assay involving the purification of EV are avoided as this is challenging in clinical settings due to variable yield, processing time and costs (Contreras-Naranjo 2017). The most robust assay(s) are useful for clinical sample validation. In one embodiment, the assay developed herein quantitates CDH17 per volume of plasma/sera. The assay may serve as an early screen. Additionally, the assay may further incorporate analysis of EV DNA or RNA for relevant gene mutations and assessment of the CDH17 EV membrane proteins for tissue of origin.

The major steps for analytical validation include:

(A) Identify CDH17 Antibodies to be Used for Efficient Capture and Detection of CDH17.

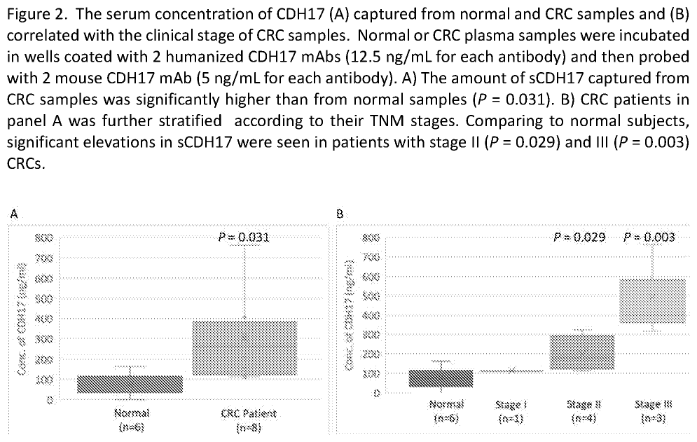

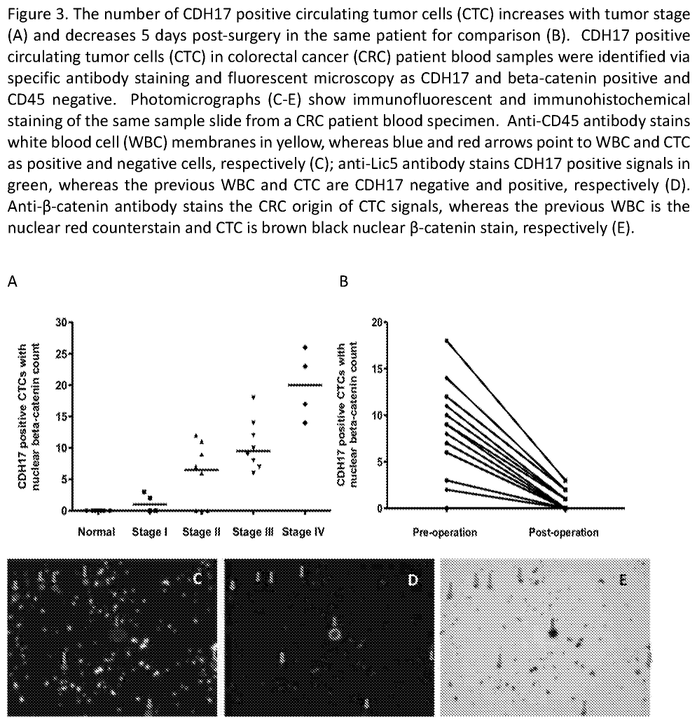

Capture antibodies. Over 400 CDH17 monoclonal antibodies were screened for their ability to capture sCDH17, CDH17 EV and total CDH17 from cancer cell culture media. Normal blood (sera/plasma) and positive patient blood were used for measuring the concentration of CDH17 in an ELISA format (FIG. 2). In addition, polyclonal antibodies and LNP may be used to capture CDH17EV. Cancer cell lines included CDH17 positive CRC (SNU-C1) and PDAC (AsCP1) lines, as well as CDH17 negative cell lines, such as SW480 and Jurkat (FIGS. 4 and 5). Capture antibodies or LNP were immobilized to microtiter plate wells (FIG. 6). To specifically measure captured sCDH17, EVs can be removed by using centrifugal filtration with a 300 kDa mwco filter (CDH17=120 kDa). To specifically measure captured CDH17EV, the washed and filtered EVs were used. As an alternative approach, captured CDH17EV were specifically measured using LNP as illustrated in FIG. 6. Captured EVs were measured by using antibodies specific to an exosome marker, such as CD63 and CD9, and/or for other EV membrane proteins not known to directly bind CDH17 (such as TROP-2) or by pre-labelling EV with a cell permeant DNA/RNA stain, such as SYTO-13. After identification of the most efficient individual capture antibodies, such as ARB101, ARB102, and 9C6 (SEQ ID No. 1-6), combinations of capture antibodies were tested in order to identify a combination with greater capture efficiency so that the sensitivity of the assay may be improved and optimized. Unique forms of CDH17 in patients' blood samples may be characterized by immunoblot and immunohistochemistry analyses (FIG. 3, low panel), whereas captured peptides may be characterized by mass spectrometry.

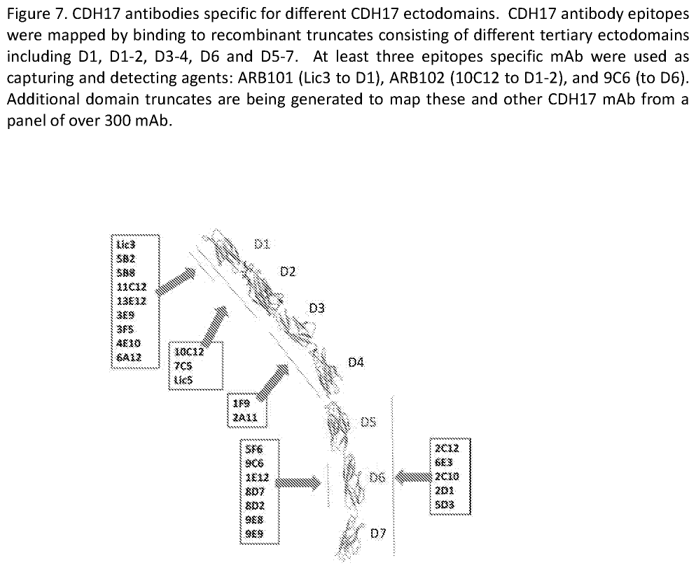

Detection antibodies. CDH17 antibodies were screened for the most sensitive detection of captured sCDH17 and CDH17EV. Using purified soluble, recombinant CDH17-Fc or CDH17his as a standard, the sensitivity of the assays at various stages of development can be determined as shown in FIG. 8. The target sensitivity for the assay is approximately 500 pg/ml or lower. Candidate capturing and detecting antibodies were among those with epitopes mapped to one or more CDH17 ectodomains, as shown in FIG. 7. These and additional epitope mapped antibodies are used to approximate the cleavage sites in sCDH17 and potentially on CDH17EV.

(B) Sample Processing; Comparison of Serum Versus Plasma.

Sets of serum and plasma samples collected from the same patient (n>10) were assayed for sCDH17 and CDH17EV to determine if one method of sample collection allows for greater CDH17 yield/detection.

(C) Generate Recombinant CDH17 Capture Antibodies to Increase Assay Efficiency.

Recombinant CDH17 was generated to characterize capture antibodies in order to increase the efficiency of these assays. To further increase capture efficiency and sensitivity, selected capture antibodies were converted to a modified recombinant probe to enable greater flexibility and functional orientation of the antibodies on substrates. On the other hands, detecting antibodies may also incorporate at least one Avi-tag for biotinylation and high affinity binding to HRP-streptavidin, or a fluorophore-streptavidin conjugate. Depending on the affinity of a key assay antibody, affinity maturation may be considered.

### EXAMPLES

### Example 1. Methods for Sample Preparation and Characterization

Exosomes were purified from culture media of CDH17 positive CRC (SNUC1) and PDAC(AsPC1) cell lines by standard differential ultracentrifugation (Bow2012). For protein detection, 10 ug of soluble exosome protein was loaded into an SDS-PAGE gel, blotted and probed with CDH17 and CD63 antibodies. For characterizing exosome, polystyrene beads (10 micron) were coated with a humanized CDH17 antibody (mh10C12) or hIgG and incubated with cell-free tumor culture media. The beads were washed and then stained with a mouse CDH17 antibody (7C5) or a CD63 antibody and anti-mlgAlex647. The antibody against exosome marker CD63 may detect 50% of CDH17 EV as it is not a marker for microvesicles. For conducting CDH17 ELISAs of cell-free media from tumor cell lines, SNUC1 culture media was passed through a 100 kDa mwco filter and tested for the level of CDH17.

Normal or CRC plasma samples and soluble CDH17 (1 ug/ml) were incubated with beads coated with a humanized CDH17 antibody or a CD68 antibody, washed and stained with a non-competitive mouse CDH17 antibody. Normal or CRC plasma samples were incubated in wells coated with a CDH17 polyclonal or a pool of three humanized CDH17 mAb, and then probed with a mouse CDH17 mAb. In some samples, CDH17 was readily captured by the polyclonal antibody. This finding indicates that the nature of CDH17 antibody plays an important role in the quality of any diagnostic method for assaying CDH17 in patient's samples or cancer cell cultures.

To increase the efficiency of capturing EVs, selected recombinant CDH17 antibodies were generated that are uniformly and functionally orientated toward the analyte. This were accomplished through site specific biotinylation of a C-terminal peptide tag (AviTag; Avidity LLC) to enable C-terminal binding to a neutravadin coated substrate. The high affinity CDH17 antibodies were anchored via a flexible linker to facilitate rapid and high avidity binding. LNP possesses a diacyl lipid (DSPE) that inserts into EV membranes, a polyethylene glycol (PEG) spacer, and a biotin tag. LNP can be bound to various substrates via biotin to capture or detect EV (Wan 2017).

The measurement of exosomes may be conducted using flow cytoflurometic, ELISA assays, and proximitry bioluminescence.

### Example 2. Methods for Charactering Circulating Tumor Cells and Extracellular Vesicles

Many methods were employed to quantify CDH17 positive samples, including histopathology, immunohistochemistry (IHC), ELISA, immunoblotting, immunofluorescence, flow cytometry, and proximity bioluminescence. In a general agreement, the levels of CDH17 seemed to be readily detectable, in particular, the levels of CDH17 positive IHC counts, serum level, or CTC counts increased as the tumor progress through each stage and decreased after surgical treatment (FIG. 3). The CTC levels in the early stage of cancer can be very low relative to the circulating exosomes originating from the tumor (Ferreira 2017). Thus, CDH17 exosomes may be released by GI tumor cells, which then became detectable in blood earlier than CTC allowing a more robust assay to detect early GI cancers, which can be used to assist in staging any GI tumors.

It has been reported that CDH17 is released from cultured GI tumor cell lines as an extracellular vesicle membrane protein (Mathivanan S. 2010, Demory B 2013. Xu R 2015). Extracellular vesicles harboring CDH17 (CDH17EV) include both exosomes (30-100 nm) and microvesicles (100-1000 nm). Indeed, CDH17EV were readily detectable in in tissue culture media of GI cancer cells as shown in FIG. 3-5. A soluble presumably shed form of CDH17 (sCDH17) with molecular weight less than 100 kDa was identified by using anti-CDH17 antibody and ELISA. Since an intact CDH17 molecule possesses 7 tertiary ectodomains (FIG. 7) and is 120 kDa (FIG. 4), this sCDH17 in tumor cell media appeared to lack Domain 6 (D6, FIG. 7) because it does not bind D6-specific antibodies. A CDH17 greater than 100 kDa was also detected in media for GI tumor cells that may be classified as CDH17EV.

The assay analysis using a few plasma samples from normal and CRC patients indicates that patient blood contains both sCDH17 and CDH17EV (FIG. 2-3). Typically, a patient's blood may have almost 1 ug/ml of CDH17, but the amount of CDH17 in normal blood is close to background. The characterization of either CDH17EV or sCDH17 in a cancer patient's blood indicates that certain antibodies that efficiently capturing CDH17 in media from cultured cancer cells may not capture CDH17 from some patients' blood. Thus, identification of CDH17 antibodies that may efficiently capture all forms of CDH17 in patients' blood is prerequisite to screening patient samples.

Although several studies have previously suggested that tumor associated CDH17 may serve as a useful and early stage biomarker, and yet a CDH17 blood assay has not been developed or validated (Lee 2010, Panarelli 2012). This may be because cleaved forms of CDH17 in patients' blood, both shed and vesicle associated, have not been characterized and appropriate capturing and detecting probes were not available. For diagnostic assay development, a panel of over 400 CDH17 antibodies have been generated with epitopes mapped to all 7 CDH17 ectodomains (see below).

Of normal individuals, the baseline CDH17 in blood may be sub-nanomolar or negligible (FIG. 1-3). Normal blood levels for other proposed markers, such as E-Cadherin, can be high and may only demonstrate a 2-fold increase in patients' blood (Weib 2011). The CDH17 assay may be further developed through the use of tissue specific antibodies to phenotype captured EV and allow for determination of the origin of the tumor (FIG. 3). The result may be further developed as a prognostic assay to guide treatment with the analysis of mutant tumor genes in captured CDH17EV. For example, KRAS and NRAS codons 12 and 13, BRAF p.V600, miRNA and other tumor driver mutant DNA/RNA in CDH17 exosomes or total EV may be analyzed for prognostic or predictive assessment (Sepulveda 2017, Ogata-Kawata 2014, Hao 2017). Efforts to develop blood based extracellular vesicle (EV) assays has recently increased with the demonstration of their ability to detect tumor associated proteins, DNA and RNA in several different platforms (Soung 2017). Finally, an assay for CDH17 blood levels will also serve as a pharmacodynamic marker for any clinical studies targeting CDH17.

Currently, no blood-based assays are available for measuring the level of CDH17 in serum or cell culture. The obstacle may be due to lack of high affinity epitope mapped CDH17 antibodies, which may be essential for quantifying the levels of sCDH17, CDH17EV, and total CDH17 with exquisite sensitivity. Alternative to such antibodies, novel lipid nanoprobe (LNP) (Wan 2017; FIG. 7) may be considered as an integrate part for capturing and detecting CDH17EV. Such an assay includes a novel modified recombinant CDH17 antibody to enable more efficient sCDH17 binding and high avidity capture of circulating CDH17EV from serum/plasma (FIG. 2-3). Moreover, the assay may be further developed to identify CDH17EV tissue of origin, and gene mutations to help select current and emerging targeted therapies for GI cancer patients.

### Example 3. The CDH17EV Assay Platform

To quantitate CDH17 EV relative to the total population of EV, EV will be captured by LNP as shown in FIG. 3. The level of CDH17 will then be quantitated using a specific and high affinity CDH17 antibody and its secondary reagents, such as anti-Ig peroxidase (ELISA), anti-Ig phycoerythrin (flow cytofluorimetry), or a CDH17 antibody conjugated bead (proximity luminescence). To quantitate captured CDH17EVs, EV will be bound to a CDH17 antibody that binds a distinct non-overlapping epitope (CDH17 mAb2) (FIG. 3). There two methods each displayed comparative advantages for quantitating CDH17EV. The first method uses the LNP probe and a secondary reagent, such as streptavidin peroxidase (SA-HRP; ELISA), strepavidin phycoerythin (SA-PE; flow cytofluorimetry), or strepavidin conjugated bead (proximity luminescence). The second method uses CDH17 mAb2 as its first line of Ab and the secondary detection reagents as described. To quantitate other proteins on CDH17 EV, CDH17 EV will be captured with a humanized CDH17 specific antibody (huCDH17 mAB). Mouse antibodies specific for the antigens (e.g. TROP2) will be allowed to bind. Their binding will be detected using anti-mouse IgHRP (ELISA), or anti-mouse-IgPE (flow cytofluorimetry). For proximity luminescence CDH17 can be captured with CDH17 mAb2 coupled bead and the second protein detected with a protein-A/G bead (proximity luminescence).

### Example 4. Select Assay Platforms and Protocols for Clinical Sample Validation

Following selection of optimal capturing and detecting antibodies in the ELISA, antibodies and LNP were used in the proximity luminescence and flow cytometry platforms. Each of the three platforms was applied to compare cancer cell culture media, positive blood samples, normal blood samples, and recombinant soluble CDH17. One or two platforms were selected for clinical sample validation assays depending on their performance, i.e. sensitivity, stability, reproducibility. Sensitivity of non-optimized assays was close to 400 pg/ml. The target criteria for assay validation includes high sensitivity (<20 pg/ml), specificity (>50-fold relative to normal sera), reproducibility, dynamic range (over 4 logs), high throughput and minimal time to perform (1-2 hours).

The primary endpoint of clinical sample validation is to have a statistically significant value that differentiates an increase in the level of sCDH17, CDH17 EV or total CDH17 in blood samples from GI cancer patients, such as a significant increase in CDH17 blood levels, change of tumor stages, and a significant decrease after treatment (FIG. 3). It is anticipated that there is constant need to optimize the standards for sCDH17, CDH17 EV and total CDH17. In this context, more than one assay platform may be employed to ensure a robust assay result for each blood sample.

The present disclosure is not to be limited in terms of the particular embodiments described in this application, which are intended as illustrations of various aspects. Many modifications and variations may be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which may, of course, vary. It is also to be understood that the terminology used herein is for the purpose of describing particular embodiments only, and is not intended to be limiting.

While the disclosure has been particularly shown and described as referenced to the embodiments thereof, those skilled in the art will understand that the foregoing and other changes in form and detail may be made therein without departing from the spirit and scope of the disclosure.

### Tables

TABLE 1
Characteristic reagents for assaying CDH17 positive EV

| EV capturing antibody | EV detecting antibody |
|---|---|
| First CDH17 Ab, e.g. | Second CDH17 Ab, e.g. 7C5, 5F6 (D6), |
| ARB101 (SEQ ID No: 1, | 9C6 (D6, SEQ ID No: 5, SEQ ID No: 6) |
| SEQ ID No: 2), | pre-labeled (or post) EV using membrane, |
| ARB102 (SEQ ID No: 3, | DNA/RNA or protein dye |
| SEQ ID No: 4), | |
| First CDH17 Ab, e.g. | Second CDH17 Ab, e.g. 7C5, 5F6 (D6), |
| ARB101 (SEQ ID No: 1, | 9C6 (D6, SEQ ID No: 5, SEQ ID No: 6) |
| SEQ ID No: 2), | LNP-biotin-strepavidin-conjugated |
| ARB102 (SEQ ID No: 3, | to HRP, fluorescent dye, etc. |
| SEQ ID No: 4) | |
| First CDH17 Ab, e.g. | Second CDH17 Ab, e.g. 7C5, 5F6 (D6), |
| ARB101 (SEQ ID No: 1, | 9C6 (D6, SEQ ID No: 5, SEQ ID No: 6) |
| SEQ ID No: 2), | Antibody specific for broad tumor |
| ARB102 (SEQ ID No: 3, | associated antigen, e.g. EGFR |
| SEQ ID No: 4) | Antibody specific for vesicles e.g. CD63 |
| | Antibody specific for tissue types |

### SEQUENCE LISTING

Examples of CDH17 capturing and detecting antibodies:

Humanized amino acid sequences of Lic3
variable heavy chain domain (ARB101, CDH17
capturing)
SEQ ID NO: 1
DIVLTQTPLSLTVSLGDQASISCRSSQSIVHSNGNTYLGWYLQ
RPGQSPKLLIYKVSNRFSGVPDRFSGSGSGTDFTLKISRVEAE
DLGVYYCFQGSHVPLTFGAGTKLELKRAD
Humanized amino acid sequences of Lic3
variable light chain domain (ARB101, CDH17
capturing)
SEQ ID NO: 2
QVQLQESGGGLVKPGGSLKLSCAASGFSFSDYYMYWVRQAPEK
RLEWVASISFDGTYTYYTDRVKGRFTISRDNAKNNLYLQ
MSSLKSEDTAMYYCARDRPAWFPYWGQGTLVTVSA
Humanized amino acid sequences of 10C12
(CDH17) variable heavy domain (ARB102,
CDH17 capturing)
SEQ ID NO: 3
EVQLVESGGGLVQPGGSLRLSCAASGFTFSSYAMSWVRQTPGK
GLEWVAVIDSNGGSTYYPDTVKDRFTISRDNSKNTLYLQMNSL
RAEDTAVYYCSSYTNLGAYWGQGTLVTVSA
Humanized amino acid sequences of 10C12
(CDH17) variable light domain (ARB102,
CDH17 capturing)
SEQ ID NO: 4
DIQMTQSPSSLSASVGDRVTITCRASQDISGYLNWLQQKPGGA
IKRLIYTTSTLDSGVPKRFSGSGSGTDFTLTISSLQSEDFATY
YCLQYASSPFTFGGGTKVEIK
Humanized amino acid sequences of 9C6
(CDH17) variable heavy domain (CDH17
detecting)
SEQ ID NO: 5 (7)
QVQLVQSGAEVKKPGASVKVSCKVSGYTFTHYWMHWVRQRPGK
GLEWMGEIDPFDSYTYYNQKFKGRVTMTVDTSSDTAYMELSSL
RSEDTAVYYCARPLPGTGWYFDVWGQGTTVTVSS
Humanized amino acid sequences of 9C6
(CDH17) variable light domain (CDH17
detecting)
SEQ ID NO: 6 (8)
EIVLTQSPTTLSLSPGERATLSCSASSSISSTYLHWYQQKPGF
PPRLLIYGTSNLASGIPACFSGSGSGTDFTLTISSLEAEDFAV
YYCQQGSSLPFTFGQGTKLEIK

What is claimed is:

1. A method for diagnosing a CDH17 positive tumor in a subject, said method comprising:
contacting a sample from the subject with a capturing antibody to provide a captured sample, wherein the capturing antibody has a binding affinity to an exosome, microvesicle or soluble CDH17 fragment;
contacting the captured sample with a detecting antibody or lipid nanoprobe (LNP) to provide a detecting sample;
determining the amount of the detecting antibody or lipid nanoprobe (LNP) in the detecting sample; and
determining the probability of the subject carrying the CDH17 positive tumor based on the amount of the detecting antibody or LNP.

2. A method for diagnosing a CDH17 positive tumor in a subject, said method comprising:
contacting a sample from the subject with a capturing antibody to provide a captured sample, wherein the capturing antibody has a binding affinity to an exosome, microvesicle or soluble CDH17 fragment;
determining the amount of captured sample; and
determining the probability of a subject having the CDH17 positive tumor based on the amount of captured sample.

3. A method for diagnosing a CDH17 positive tumor in a subject, said method comprising:
labeling a sample from the subject with a florescent DNA/RNA stain to provide a labeled sample;
contacting the labeled sample with a capturing antibody to provide a captured sample, wherein the capturing antibody has a binding affinity to an exosome, microvesicle or soluble CDH17 fragment;
determine the amount of captured sample; and
determining the probability of a subject having the CDH17 positive tumor based on the amount of captured sample.

4. The method of claim 1, wherein the capturing antibody comprises a monoclonal antibody having a binding affinity to CDH17.

5. The method of claim 1, wherein the capturing antibody comprises an amino acid sequence having at least 98% homology with SEQ ID NO. 1-6.

6. The method of claim 1, wherein the capturing antibody comprises a monoclonal antibody having a binding affinity to CD9, CD63, CD81, CD45 or a combination thereof.

7. The method of claim 1, wherein the detecting antibody comprises an antibody having a binding affinity to CDH17, TROP2, CD63, CD9, CD81, CD45, a tumor marker, a tissue marker, or a combination thereof.

8. The method of claim 1, wherein the contacting the captured sample consists of contacting the captured sample with a lipid nanoprobe (LNP).

9. The method of claim 1, wherein said CDH17 positive tumor comprises a cancer of the gastrointestinal system.

10. (canceled)

11. The method of claim 1, wherein said sample comprises peripheral blood, serum, plasma, urine, bone marrow, pleural and peritoneal fluid, or intestinal fluid.

12. (canceled)

13. (canceled)

14. The method of claim 2, wherein the capturing antibody comprises a monoclonal antibody having a binding affinity to CDH17.

15. The method of claim 2, wherein the capturing antibody comprises an amino acid sequence having at least 98% homology with SEQ ID NO. 1-6.

16. The method of claim 2, wherein the capturing antibody comprises a monoclonal antibody having a binding affinity to CD9, CD63, CD81, CD45 or a combination thereof.

17. The method of claim 2, wherein the detecting antibody comprises an antibody having a binding affinity to CDH17, TROP2, CD63, CD9, CD81, CD45, a tumor marker, a tissue marker, or a combination thereof.

18. The method of claim 2, wherein said CDH17 positive tumor comprises a cancer of the gastrointestinal system.

19. The method of claim 3, wherein the capturing antibody comprises a monoclonal antibody having a binding affinity to CDH17.

20. The method of claim 3, wherein the capturing antibody comprises an amino acid sequence having at least 98% homology with SEQ ID NO. 1-6.

21. The method of claim 3, wherein the capturing antibody comprises a monoclonal antibody having a binding affinity to CD9, CD63, CD81, CD45 or a combination thereof.

22. The method of claim 3, wherein the detecting antibody comprises an antibody having a binding affinity to CDH17, TROP2, CD63, CD9, CD81, CD45, a tumor marker, a tissue marker, or a combination thereof.

23. The method of claim 3, wherein said CDH17 positive tumor comprises a cancer of the gastrointestinal system.
